(19) United States
(12) Patent Application Publication
(10) Pub. No.: US 2024/0423838 A1
(43) Pub. Date: Dec. 26, 2024

(54) Testing and Calibrating an Automatic Ophthalmic Surgical System

(71) Applicant: BELKIN VISION LTD., Yavne (IL)

(72) Inventors: Zachary Shane Sacks, Modiin (IL); Vladimir Horesh, Yavne (IL)

(21) Appl. No.: 18/707,886

(22) PCT Filed: Dec. 1, 2022
(86) PCT No.: PCT/IB2022/061642

§ 371 Date: May 7, 2024

Related U.S. Application Data
(60) Provisional application No. 63/286,048, filed on Dec. 5, 2021.

(51) Int. Cl. A61F 9/008; A61B 34/32; A61B 90/00

(52) U.S. Cl. CPC A61F 9/00819; A61B 34/32; A61B 90/361; A61F 2009/00844

(57) ABSTRACT

A system (20) includes a radiation source (48) configured to emit beams (52) of radiation, one or more beam-directing elements (49) configured to direct the beams, a card (22) configured to undergo a change in appearance at sites on the card on which the beams impinge, a camera (54) configured to acquire one or more images of the card, and a controller (44). The controller is configured to process the images and to control the beam-directing elements, in response to processing the images, so as to direct the beams at one or more target points (74) in a field of view (72) of the camera, thereby causing the appearance of the card to change at one or more irradiated locations (76) on the card. Other embodiments are also described.

### CROSS-REFERENCE TO RELATED APPLICATIONS

The present application claims the benefit of U.S. Provisional Appl. No. 63/286,048, filed Dec. 5, 2021, whose disclosure is incorporated herein by reference.

### FIELD OF THE INVENTION

The present invention is related to automated laser eye surgery, such as automated trabeculoplasty, iridotomy, and capsulotomy procedures.

### BACKGROUND

Co-assigned U.S. Pat. No. 11,382,794 to Sacks et al. describes a system including a radiation source and a controller. The controller is configured to display a live sequence of images of an eye of a patient and, while displaying the sequence of images, cause the radiation source to irradiate the eye with one or more aiming beams, which are visible in the images. The controller is further configured to receive a confirmation input from a user subsequently to causing the radiation source to irradiate the eye with the aiming beams, and to treat the eye, in response to receiving the confirmation input, by causing the radiation source to irradiate respective target regions of the eye with a plurality of treatment beams.

Co-assigned US Patent Application Publication 2022/0125641 to Sacks and Belkin describes a system including a laser, configured to irradiate a target site in an iris of an eye, and a controller. The controller is configured to identify, in one or more images of at least part of the iris, an indication of fluid flow through the target site, and in response to identifying the indication, inhibit the laser from further irradiating the target site.

Co-assigned International Patent Application Publication WO/2022/018525 to Sacks and Belkin describes a system including a radiation source and a controller. The controller is configured to define a treatment zone on a capsule of an eye of a subject, and to form an opening in the capsule, subsequently to defining the treatment zone, by irradiating multiple target regions within the treatment zone in an iterative process that includes, during each one of multiple iterations of the process, acquiring an image of at least part of the capsule, designating one of the target regions based on the acquired image, and causing the radiation source to irradiate the designated target region.

U.S. Pat. No. 7,456,949 describes methods, systems, and apparatus for calibrating a laser ablation system, such as an excimer laser system for selectively ablating a cornea of a patient's eye, and facilitates alignment of eye tracking cameras that measure a position of the eye during laser eye surgery. A calibration and alignment fixture for a scanning laser beam delivery system having eye tracking cameras may include a structure positionable in a treatment plane. The structure having a feature directing laser energy incident thereon to a calibration energy sensor, at least one reference-edge to determine a characteristic of the laser beam (shape, dimensions, etc.), and an artificial pupil to determine alignment of the eye tracking cameras with the laser system.

### SUMMARY OF THE INVENTION

There is provided, in accordance with some embodiments of the present invention, a system including a radiation source configured to emit beams of radiation, one or more beam-directing elements configured to direct the beams, a card configured to undergo a change in appearance at sites on the card on which the beams impinge, a camera configured to acquire one or more images of the card, and a controller. The controller is configured to process the images and to control the beam-directing elements, in response to processing the images, so as to direct the beams at one or more target points in a field of view (FOV) of the camera, thereby causing the appearance of the card to change at one or more irradiated locations on the card.

In some embodiments, the card includes a polymer.

In some embodiments, the card includes transparent glass.

In some embodiments, the card includes a light-emitting material configured to undergo the change in appearance by emitting light in response to the beams of radiation.

In some embodiments, the change in appearance includes a change in color.

In some embodiments, the card includes a photosensitive dye configured to undergo the change in color in response to the beams of radiation.

In some embodiments, the card includes a temperature-sensitive material configured to undergo the change in color in response to being heated by the beams of radiation.

In some embodiments, the card is configured to undergo the change in appearance by virtue of the beams forming respective holes at the sites.

In some embodiments, the controller is further configured to move the camera with respect to the card between acquisitions of the images.

In some embodiments, the system further includes a jig configured to move the card with respect to the camera between acquisitions of the images.

In some embodiments, the system further includes:
an optical unit; and
an XYZ stage unit including a control mechanism,
and the optical unit includes the camera and is mounted onto the XYZ stage unit so as to be moveable by a user, using the control mechanism, between acquisitions of the images.

In some embodiments,
the card includes one or more markings, and
for each of the images, the controller is configured to:
   identify at least one of the markings in the image, and
   control the beam-directing elements in response to identifying the at least one of the markings.

In some embodiments, for each of the images, the controller is configured to control the beam-directing elements so as to direct a respective one of the beams at one of the identified markings.

In some embodiments,
the markings include an iris-shaped marking that simulates a human iris with respect to shape, and
for each of the images, the controller is configured to:
   identify the iris-shaped marking in the image,
   compute a respective one of the target points with reference to the iris-shaped marking, and
   control the beam-directing elements so as to direct a respective one of the beams at the computed one of the target points.

In some embodiments,
a background of the card surrounding the iris-shaped marking has a background appearance, and
at at least one location along a perimeter of the iris-shaped marking, a transition between the background appearance and an appearance of the iris-shaped marking occurs over at least 0.1 mm.

In some embodiments,
a background of the card surrounding the iris-shaped marking has a background appearance, and
at at least one location along a perimeter of the iris-shaped marking, a transition between the background appearance and an appearance of the iris-shaped marking occurs over less than 0.1 mm.

In some embodiments, the controller is further configured to:
identify the irradiated locations in another image of the card,
in response to identifying the irradiated locations, compute a distance between one of the irradiated locations and the target point at which the beam that impinged on the irradiated location was directed, and
communicate an output in response to the distance.

In some embodiments, the controller is further configured to:
prior to controlling the beam-directing elements, display another image of the card with one or more overlaid target-markers,
receive, from a user, an adjustment of respective positions of the overlaid target-markers, and
define the target points in response to the adjusted positions.

In some embodiments, the controller is further configured to display another image of the card, which shows the irradiated locations, with one or more overlaid target-markers at the target points.

In some embodiments, the card includes an iris-shaped marking that simulates a human iris with respect to shape, and the overlaid target-markers include an arced target-marker surrounding the iris-shaped marking and passing through the target points.

There is further provided, in accordance with some embodiments of the present invention, a method including coupling a card, which is configured to undergo a change in appearance at sites on the card on which beams of radiation impinge, to a jig, and by inputting a command to a controller, initiating a testing procedure during which the controller processes one or more images of the card acquired by a camera while the card is coupled to the jig, and in response to processing the images, controls one or more beam-directing elements so as to direct the beams at one or more target points in a field of view (FOV) of the camera, thereby causing the appearance of the card to change at one or more irradiated locations on the card.

The present invention will be more fully understood from the following detailed description of embodiments thereof, taken together with the drawings, in which:

### BRIEF DESCRIPTION OF THE DRAWINGS

FIG. 1 is a schematic illustration of an ophthalmic surgical system, in accordance with some embodiments of the present invention;

FIG. 2 is a schematic illustration of a card, in accordance with some embodiments of the present invention;

FIG. 3 is a schematic illustration of a jig holding a card, in accordance with some embodiments of the present invention;

FIG. 4 is a schematic illustration of an image for use in defining target points, in accordance with some embodiments of the present invention; and

FIG. 5 is a schematic illustration of an image showing irradiated locations on a card, in accordance with some embodiments of the present invention.

### DETAILED DESCRIPTION OF EMBODIMENTS

### Overview

Some automatic ophthalmic surgical systems, such as those described in the co-assigned patent and publications cited in the Background, comprise a controller configured to identify target points on an eye by processing images of the eye, and to direct beams of radiation at the identified target points. For such systems, it may be important to test, from time to time, the accuracy with which the target points are identified and the radiation beams are directed. If the accuracy is insufficient, the system may require calibration.

Hypothetically, a beam profiler could be used to test and calibrate the system. However, a beam profiler may be too small to accommodate a typical pattern of target points irradiated during a surgical procedure. Alternatively or additionally, a beam profiler may be unable to accommodate the typical intensity of the radiation beams. Moreover, the appearance of a beam profiler may be very different from the appearance of an eye, such that the beam profiler may not facilitate a proper test of the image-processing functionality of the controller.

Hence, embodiments of the present invention provide a card for use in testing and calibrating an automatic ophthalmic surgical system. The card is configured to undergo a change in appearance at sites on the card on which the beams of radiation impinge; for example, the beams may change the color of the card or form holes in the card. Hence, following the irradiation of one or more sites on the card, it may be ascertained (automatically or manually) whether these sites coincide with the intended target points. If not, the system may be calibrated, by iteratively adjusting the system and repeating the test (using one or more additional cards if required) until the desired accuracy is achieved.

Advantageously, the card may accommodate a typical pattern of target points and a typical beam intensity. Furthermore, the card may comprise a simulated iris, optionally with a simulated limbus, such that an image of the card may appear similar to an image of an eye. Thus, in testing the accuracy of the system, a simulated surgical procedure may be performed on the card, as if the card were an eye.

### System Description

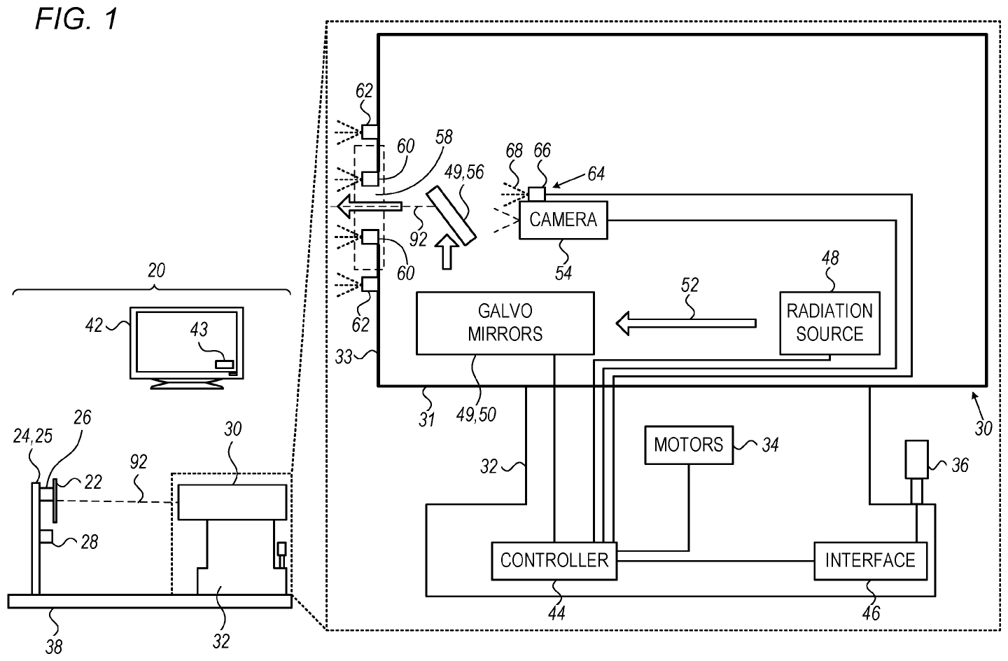

Reference is initially made to FIG. 1, which is a schematic illustration of an ophthalmic surgical system 20, in accordance with some embodiments of the present invention.

System 20 comprises a radiation source 48 configured to emit beams 52 of radiation. For example, radiation source 48 may comprise a laser, such as a frequency-doubled passively or actively Q-switched Nd:YAG laser, configured to emit beams of laser radiation. Alternatively or additionally to a laser, the radiation source may comprise an array of light-emitting diodes (LEDs), an array of laser diodes, and/or an electric flash-lamp.

In some embodiments, beams 52 comprise visible light. Alternatively or additionally, the beams may comprise non-visible electromagnetic radiation, such as microwave radiation, infrared radiation, X-ray radiation, gamma radiation, or ultraviolet radiation. In some embodiments, the wavelength of the beams is between 200 and 11000 nm, e.g., 500-850 nm, such as 520-540 nm, e.g., 532 nm. Typically, the energy of each beam is between 0.1 and 4 mJ, such as between 0.3 and 2.6 mJ. The spatial profile of each beam may be elliptical (e.g., circular), square, or of any other suitable shape. The intensity profile of each beam may be Gaussian, super-Gaussian, or top-hat along any one or more cross-sections of the beam.

System 20 further comprises one or more beam-directing elements 49 configured to direct the beams of radiation. Beam-directing elements 49 may comprise, for example, one or more galvo mirrors 50, which may be referred to, collectively, as a “galvo scanner,” and/or a beam combiner 56. Each beam may deflect off of galvo mirrors 50 toward beam combiner 56, and then deflect off of the beam combiner along a beam path 92.

System 20 further comprises a controller 44 and a camera 54. Controller 44 is configured to process images acquired by camera 54, and in response thereto, to control beam-directing elements 49 so as to direct beams 52 at any desired target points within the field of view (FOV) of the camera. In particular, before the emission of each beam 52 from radiation source 48, and/or while the beam is being emitted, controller 44 may adjust the position, orientation, size, and/or shape of one or more of the beam-directing elements such that the beam-directing elements direct the beam at the desired target point.

In general, camera 54 may comprise one or more imaging sensors of any suitable type(s), such as a charge-coupled device (CCD) sensor, a complementary metal-oxide-semiconductor (CMOS) sensor, an optical coherence tomography (OCT) sensor, and/or a hyperspectral image sensor. Using the sensors, the camera may acquire two-dimensional or three-dimensional images of any suitable type, such as monochrome images, color images (based, for example, on three color frames), multispectral images, hyperspectral images, optical coherence tomography (OCT) images, or images produced by fusing multiple images of different respective types.

In some embodiments, the camera is positioned behind beam combiner 56, such that the camera receives light via the beam combiner. In other embodiments, the camera is offset from the beam combiner.

Typically, system 20 comprises an optical unit 30 comprising radiation source 48, camera 54, and beam-directing elements 49. Typically, optical unit 30 comprises an optical bench, and the radiation source and beam-directing elements are coupled to the optical bench. Optical unit 30 may further comprise a front face 33 shaped to define an opening 58, or comprising an exit window, through which beams 52 are directed. For example, optical unit 30 may comprise an encasement 31, which at least partially encases the optical bench and comprises front face 33. Alternatively, front face 33 may be attached to, or may be an integral part of, the optical bench.

Typically, optical unit 30 is mounted onto an XYZ stage unit 32 comprising a control mechanism 36, such as a joystick, with which a user of system 20 may adjust the position and orientation of the optical unit.

For example, XYZ stage unit 32 may comprise one or more motors 34, and control mechanism 36 may be connected to interface circuitry 46. As the user manipulates the control mechanism, interface circuitry 46 may translate this activity into appropriate signals and output these signals to controller 44. In response to the signals, the controller may control motors 34. Alternatively, XYZ stage unit 32 may be controlled manually by manipulating the control mechanism; in such embodiments, the XYZ stage unit may comprise a set of gears and rollers instead of motors 34.

In some embodiments, optical unit 30 further comprises a light source 66, which is configured to function as a fixation target 64 by transmitting visible fixation light 68. Light source 66 may comprise a light emitter, such as a light emitting diode (LED), or a reflector configured to reflect light emitted from a light emitter.

In some embodiments, optical unit 30 further comprises one or more illumination sources 60 comprising, for example, one or more LEDs, such as white-light or infrared LEDs. In such embodiments, controller 44 may cause illumination sources 60 to flash while camera 54 acquires an image, thereby facilitating the acquisition of the image. (For ease of illustration, the electrical connection between controller 44 and illumination sources 60 is not shown explicitly in FIG. 1.) As shown in FIG. 1, illumination sources 60 may be coupled to front face 33; for example, the illumination sources may be arranged in a ring surrounding opening 58.

To facilitate positioning the optical unit, the optical unit may further comprise a plurality of beam emitters 62 (comprising, for example, respective laser diodes), which are configured to emit a plurality of triangulating range-finding beams, e.g., as described in U.S. Pat. No. 11,382,794 to Sacks et al., whose disclosure is incorporated herein by reference. As shown in FIG. 1, beam emitters 62 may be coupled to front face 33.

Typically, system 20 further comprises a display 42, configured to display images acquired by the camera and/or other output. Display 42 may be attached to optical unit 30 or belong to a separate device, such as a computer monitor, disposed at any suitable location.

In some embodiments, display 42 comprises a touch screen, and the user inputs commands to the system via the touch screen. Alternatively or additionally, system 20 may comprise any other suitable input devices, such as a keyboard or a mouse.

In some embodiments, display 42 is connected directly to controller 44 over a wired or wireless communication interface. In other embodiments, display 42 is connected to controller 44 via an external processor, such as a processor belonging to a standard desktop computer.

In some embodiments, as shown in FIG. 1, controller 44 is disposed within XYZ stage unit 32. In other embodiments, controller 44 is disposed externally to the XYZ stage unit. Alternatively or additionally, the controller may cooperatively perform at least some of the functionality described herein with another, external processor.

System 20 further comprises a card 22. At any suitable intervals (e.g., once a day, before any surgical procedures are performed on that day), card 22 may be used in a testing procedure for verifying the calibration of beam-directing elements 49 and the image-processing functionality of controller 44, as further described below with reference to the subsequent figures.

Prior to the testing procedure, a user couples card 22 to a jig 24. Subsequently, the user initiates the testing procedure, e.g., by touching or clicking on a button 43 displayed on display 42, or by inputting a command to the controller in any other way, such that controller 44 executes the testing procedure while the card is held by jig 24.

In some embodiments, as shown in FIG. 1, jig 24 and XYZ stage unit 32 are both mounted onto a surface 38, such as a tray or tabletop. In other embodiments, the XYZ stage unit is mounted onto surface 38, and the jig is attached to the XYZ stage unit.

In some embodiments, jig 24 is stationary. For example, jig 24 may comprise a headrest 25, comprising a forehead rest 26 and a chinrest 28, on which the patient rests his head during the surgical procedure. During the testing procedure, card 22 may be mounted onto headrest 25, e.g., onto forehead rest 26 as shown in FIG. 1.

In other embodiments, jig 24 is non-stationary, as further described below with reference to FIG. 3.

Typically, during the testing procedure, the card is distanced from the radiation source such that a dimension (e.g., diameter) of the spot size of each beam 52 on the card is between 0.3 and 0.5 mm. As noted below with reference to FIG. 3, the distance of the card from the radiation source may vary during the testing procedure.

In some embodiments, at least some of the functionality of controller 44, as described herein, is implemented in hardware, e.g., using one or more fixed-function or general-purpose integrated circuits, Application-Specific Integrated Circuits (ASICs), and/or Field-Programmable Gate Arrays (FPGAs). Alternatively or additionally, controller 44 may perform at least some of the functionality described herein by executing software and/or firmware code. For example, controller 44 may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the controller in electronic form, over a network, for example. Alternatively or additionally, the program code and/or data may be provided and/or stored on non-transitory tangible media, such as magnetic, optical, or electronic memory. Such program code and/or data, when provided to the controller, produce a machine or special-purpose computer, configured to perform the tasks described herein.

In some embodiments, the controller comprises a system on module (SOM), such as the Varisite™ DART-MX8M.

### The Card

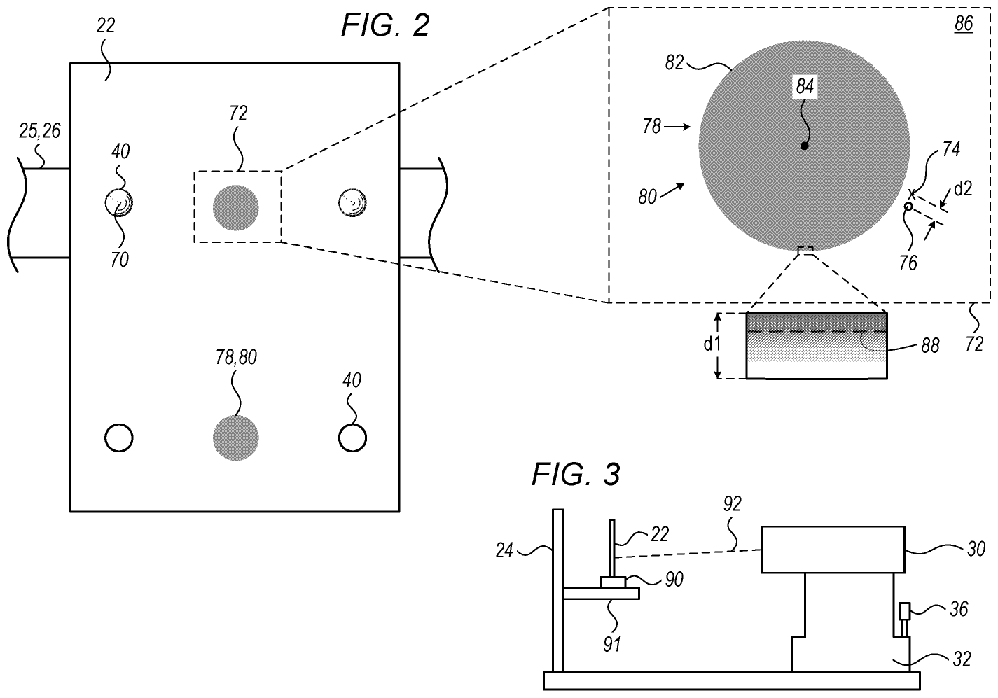

Reference is now made to FIG. 2, which is a schematic illustration of card 22, in accordance with some embodiments of the present invention.

Card 22 is configured to undergo a transient or permanent change in appearance at sites on the card on which beams 52 (FIG. 1) impinge.

In some embodiments, the change in appearance includes a change in color. For example, the card may change color by undergoing a chemical reaction or by any of the other mechanisms described below. Alternatively or additionally, the card may comprise a photosensitive dye configured to undergo the change in color in response to the beams of radiation. The dye may be integrated into the material of the card or coated onto the material.

In other embodiments, the card is configured to undergo the change in appearance by virtue of the beams forming respective holes at the sites.

In some embodiments, card 22 comprises transparent glass or a polymer such as acrylonitrile butadiene styrene (ABS), polyethylene, or polyvinyl chloride (PVC). In such embodiments, the change in appearance is typically due to a chemical reaction caused by the beams of radiation. The chemical reaction may include, for example, pyrolysis, foaming, bleaching, carbonization, or (for transparent glass or a transparent polymer) the formation of microcavities by photoablation.

In other embodiments, the card comprises a temperature-sensitive material, such as liquid crystal or thermal paper, configured to change color in response to being heated by the beams of radiation.

In yet other embodiments, the card comprises a light-emitting material configured to undergo the change in appearance by emitting light (e.g., via fluorescence or a multiphoton absorption process) in response to the beams of radiation. For example, the card may comprise a material found in ultraviolet (UV) or infrared (IR) laser sensor cards.

The card may be rectangular or may have any other suitable shape. Typically, the surface area of the card is between 50 and 150 cm². For example, for a rectangular card, the length of the card may be between 8 and 10 cm and the width of the card may be between 6 and 9 cm. Typically, the thickness of the card is between 0.01 and 1 mm.

During the testing procedure, camera 54 (FIG. 1) focuses on the card such that the FOV 72 of the camera contains at least part of the card. In response to processing images of the card acquired by the camera, controller 44 (FIG. 1) identifies one or more target points 74 in FOV 72. For each target point 74, the controller controls beam-directing elements 49 (FIG. 1) so as to direct a beam of radiation at the target point, thereby causing the appearance of the card to change at an irradiated location 76 on the card. (Typically, the size of each irradiated location 76 is within±10% of the spot size of the beam on the card.) Typically, at any given time during the testing procedure, FOV 72 contains all of the irradiated locations 76 that have been created up until the given time.

As described above with reference to FIG. 1, card 22 may be mounted onto headrest 25 during the testing procedure. For example, card 22 may be shaped to define one or more holes 40, and forehead rest 26 may comprise respective knobs 70 configured to hold the card to the forehead rest by fitting through holes 40. Alternatively, the card may be coupled to the forehead rest, or to any other portion of the headrest, by any other coupling mechanism such as a clip, a magnet, or a hook-and-loop fastener.

Typically, card 22 comprises one or more markings 78 for testing the image-processing functionality of the controller, e.g., by functioning as targets or as iris simulators as described immediately below. In some embodiments, markings 78 are printed onto background 86. In other embodiments, the markings comprise stickers stuck onto the background.

For each image processed by the controller, the controller identifies at least one marking 78 in the image and controls the beam-directing elements in response to identifying the marking.

For example, for each processed image, the controller may control the beam-directing elements so as to direct a beam at one of the identified markings. In other words, the markings may function as targets for the controller. If irradiated locations 76 coincide with the markings, it may be ascertained that the controller is processing the images properly and that the beam-directing elements are calibrated correctly. (Typically, in such embodiments, markings 78 are much smaller than indicated in FIG. 2; for example, the size of each marking may be approximately equal to the spot size of each beam on the card.)

Alternatively or additionally, as shown in FIG. 2, markings 78 may include an iris-shaped marking 80, which, as further described below, simulates the iris—and, optionally, the limbus—of a human eye. For each of the processed images, the controller may identify iris-shaped marking 80 in the image and then compute a respective one of target points 74 (i.e., compute the coordinates, in FOV 72, of a respective one of the target points) with reference to the iris-shaped marking. For example, the controller may identify the edge 82 of the iris-shaped marking using any suitable edge-detection algorithm, compute the coordinates of a center point 84 at the center of edge 82, and then compute the target point by adding a predefined offset to point 84. Subsequently, the controller may control the beam-directing elements so as to direct a beam at the computed target point. If the resulting irradiated location 76 coincides with the target point, it may be ascertained that the beam-directing elements are calibrated correctly. If, in addition, the target point appears to be at the correct location relative to the iris-shaped marking, it may be ascertained that the controller is processing the images properly.

Iris-shaped marking 80 simulates a human iris with respect to shape. For example, the iris-shaped marking may be elliptical. (In such embodiments, the lengths of the major and minor axes of the iris-shaped marking may be within 10% of one another, e.g., the lengths may equal one another such that the iris-shaped marking is circular.) Alternatively, the shape of the iris-shaped marking may deviate from an ellipse, the size of the deviation being within the range of deviations exhibited in human irises.

In addition, the iris-shaped marking may simulate an iris with respect to size. For example, for an ellipse, the length of the major axis (or, in the case of a circle, the diameter) of the iris-shaped marking may be between 8 and 13 mm.

Alternatively or additionally, iris-shaped marking 80 may simulate an iris with respect to color. In addition, the background 86 of the card surrounding the iris-shaped marking may be colored white, so as to simulate a sclera.

Alternatively, the color of the iris-shaped marking may be different from that of an iris, and/or the color of background 86 may be different from that of a sclera.

For example, in some embodiments, the controller processes only a single frame of the image, such as the red (“R”) frame. In such embodiments, even colors that are dissimilar to those of an iris and sclera may be selected, provided that the pixel values in the processed frame are similar to those that would appear in the processed frame of an image of an eye. For example, the colors may be selected such that, in the processed frame, the background pixel values are between 105 and 145 (the maximum pixel value being 255), and/or the pixel values of the iris-shaped marking are between 40 and 60, regardless of the pixel values in the other frames.

Alternatively, even the pixel values in the processed frame may be dissimilar to those that would appear in the processed frame of an image of an eye. For example, the iris-shaped marking may be black, such that the pixel values of the iris-shaped marking are approximately zero.

In some embodiments, to enhance the testing of the controller's image-processing functionality, iris-shaped marking 80 also simulates a limbus of an eye. In particular, at at least one location along the perimeter of the iris-shaped marking (e.g., along the entire perimeter), the transition between the appearance of background 86 and the appearance of the iris-shaped marking (e.g., the transition between the color and/or brightness of the background and the color and/or brightness of the iris-shaped marking) is relatively gradual. For example, the transition may occur over a distance d1 of at least 0.1 mm, such as between 0.1 and 4 mm. In some embodiments, the gradual transition is achieved by grayscale printing of the iris-shaped marking.

In such embodiments, typically, the controller identifies a closed curve 88 passing through the points of maximum gradient in the image, and then computes edge 82 by smoothing curve 88 or by fitting a predefined shape (e.g., an ellipse, such as a circle) to curve 88.

Alternatively or additionally, to help model extreme cases, the transition is relatively abrupt at at least one location along the perimeter of the iris-shaped marking; for example, the transition may occur over less than 0.1 mm.

Alternatively or additionally to a simulated limbus, other features of iris-shaped marking 80 may increase the resemblance of the iris-shaped marking to an iris. Such features may include, for example, a simulated pupil at the center of the iris-shaped marking and/or simulated blood vessels running through the iris-shaped marking.

As noted above, card 22 may comprise multiple markings 78. For example, the card may comprise multiple iris-shaped markings 80, such that the card can be used for multiple testing procedures.

In some embodiments, prior to the firing of each beam 52 at a target point, the controller causes the radiation source to fire an aiming beam at the target point. By virtue of differing from beam 52 with respect to wavelength and/or intensity, the aiming beam does not cause the appearance of the card to change; rather, the aiming beam merely reflects off the card. By processing an image of the card so as to locate the reflection, the controller verifies that the reflection is at the approximate location of the target point. In response to this verification, the controller fires beam 52.

Alternatively or additionally, prior to the firing of each beam 52, the controller may process a feedback signal from an encoder of at least one beam-directing element. In response to verifying, based on the feedback signal, that the beam-directing element is properly positioned, oriented, sized, and/or shaped, the controller may fire beam 52.

Reference is now made to FIG. 3, which is a schematic illustration of jig 24 holding card 22, in accordance with some embodiments of the present invention.

In some embodiments, jig 24 is non-stationary, and is configured to move the card with respect to the camera (e.g., so as to simulate movement of an eye) between acquisitions of the images by the camera. This movement, which may have up to six degrees of freedom, causes markings 78 (FIG. 2) to move and/or change size within FOV 72, thus further testing the image-processing functionality of the controller by requiring the controller to track the movement of the markings. For example, jig 24 may comprise a stage 90 configured to move along a stationary platform 91 while holding the card.

For embodiments in which jig 24 is stationary (e.g., as in FIGS. 1-2), the controller may be configured to move the camera with respect to the card—e.g., by controlling motors 34 (FIG. 1) so as to move optical unit 30 with respect to the card—between acquisitions of the images by the camera. Alternatively, using control mechanism 36, the user may move the optical unit between the acquisitions of the images. In either case, the movement of the camera with respect to the card, which may have up to six degrees of freedom, causes the markings to move and/or change size within FOV 72, thereby testing the image-processing functionality of the controller.

### Target Definition

Typically, at the start of the testing procedure, the controller computes multiple target points. In some embodiments, the target points lie along an arced path, such as an elliptical (e.g., circular) path.

As described above with reference to FIG. 2, in some embodiments, card 22 comprises markings 78 that function as targets. In such embodiments, each target point 74 (FIG. 2) is defined, by the controller, as the point in FOV 72 at which one of the markings lies.

In other embodiments, each target point is computed, by the controller, by adding a predefined offset to a reference point, such as center point 84, that is located using image processing. By way of example, the controller may compute K target points lying along a circular path, each k^{th} one of the target points having the coordinates (x0(t)+Rcosθₖ, y0(t)+Rsinθₖ), where:
x0(t) and y0(t) are the x-coordinate and y-coordinate of the reference point in the FOV of the camera,
R is the predefined radius of the circular path, and

$${θ}_{k} = 2 π k / K for k = 0 … K - 1.$$

(The coordinates of the reference point are expressed as functions of time, given that these coordinates may change due to movement of the card or of the camera.)

In such embodiments, the offsets may be predefined by the controller; for example, for a circular path of target points, the controller may predefine the variables R and K, which determine the offsets.

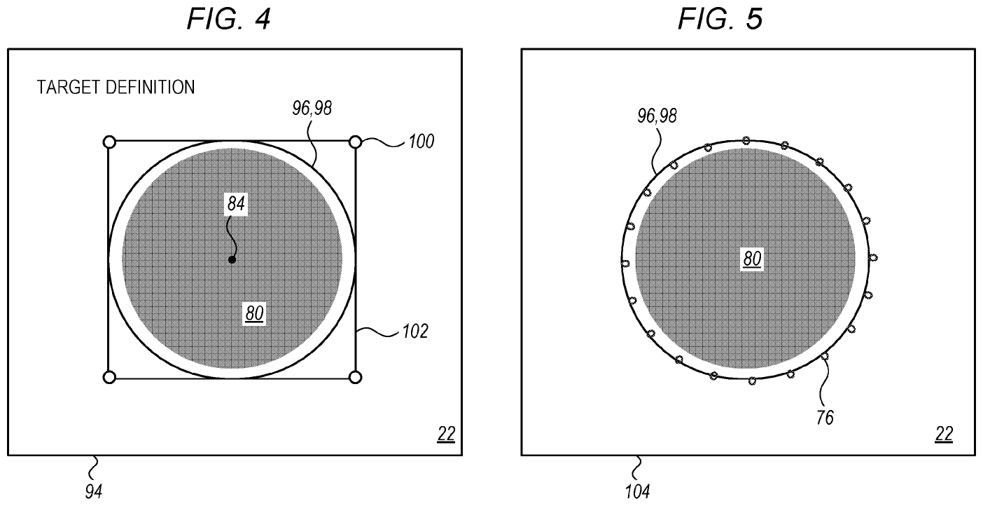

Alternatively, the offsets may be defined by the user prior to the testing procedure. In this regard, reference is now made to FIG. 4, which is a schematic illustration of an image 94 of card 22, acquired by camera 54 (FIG. 1), for use in defining target points, in accordance with some embodiments of the present invention.

In some embodiments, prior to the testing procedure, the controller displays image 94 (e.g., on display 42 (FIG. 1)) with one or more overlaid target-markers 96 marking potential locations of the target points. The controller then receives, from the user, any desired adjustment of the respective positions of the overlaid target-markers.

For example, the controller may overlay a single continuous target-marker marking a potential path along which the target points may lie. As a specific example, for embodiments in which card 22 comprises iris-shaped marking 80, overlaid target-markers 96 may include an arced (e.g., elliptical, such as circular) target-marker 98 surrounding iris-shaped marking 80 at a predefined distance from the edge of the iris-shaped marking. Using a mouse or any other suitable input device, the user may adjust this distance, e.g., by dragging the corners 100 of a rectangle (e.g., a square) 102 circumscribing target-marker 98.

Optionally, the user may also set the number of target points, e.g., the number K described above.

After defining the offsets (and, optionally, setting the number of target points), the user initiates the testing procedure. Subsequently, the controller defines the target points in response to the adjusted positions of target-markers 96 (and, optionally, in response to the desired number of target points). For example, based on the adjusted position of a circular target-marker 98, the controller may calculate R as the distance from target-marker 98 to center point 84, and then use R to compute the coordinates of each target point as described above.

### Assessing Test Results

Reference is again made to FIG. 2.

In some embodiments, following the firing of one or more beams at card 22, the controller identifies irradiated locations 76 in an image of the card acquired by the camera. In response to identifying the irradiated locations, the controller computes a distance d2 between one of the irradiated locations and the corresponding target point 74, i.e., the target point at which the beam that impinged on the irradiated location was directed.

For example, using a spot-detection algorithm, the controller may detect the center of the irradiated location. Alternatively, the controller may detect the edge of the irradiated location, and then compute the center based on the edge. In addition, the controller may process the image so as to identify the current coordinates (x0′, y0′) of the reference point, such as center point 84. Subsequently, based on the coordinates of the reference point, the controller may calculate the current coordinates of the target point at which the beam was directed; for example, for a circular target path, the controller may add Rcosθₖ to x0′ and Rsinθₖ to y0′. Subsequently, the controller may calculate the distance d2 between these latter coordinates and the center of the irradiated location.

In response to distance d2 (and, optionally, at least one additional such distance), the controller communicates an output, e.g., by displaying an appropriate message on display 42 (FIG. 1). For example, the controller may compare d2 (or a statistic, such as an average or a maximum, of multiple such distances for multiple irradiated locations) to a predefined threshold. If d2 (or the statistic) exceeds the threshold, the controller may communicate an output (e.g., display a message) indicating that the beam-directing elements must be calibrated before a surgical procedure is performed.

In other embodiments, the user manually assesses the test results. In this regard, reference is now made to FIG. 5, which is a schematic illustration of an image 104 showing irradiated locations 76 on card 22, in accordance with some embodiments of the present invention. In some embodiments, the controller is configured to display (e.g., on display 42 (FIG. 1)) image 104, which was acquired by the camera and shows irradiated locations 76, with one or more overlaid target-markers 96 at the target points. (In other words, target-markers 96 are overlaid at the current coordinates of the target points in the FOV of the camera.) For example, the controller may overlay a single continuous target-marker 98 passing through each of the target points. As a specific example, for embodiments in which card 22 comprises iris-shaped marking 80, overlaid target-markers 96 may include an arced (e.g., circular) target-marker 98 surrounding iris-shaped marking 80 and passing through the target points. In other words, the target points may lie along an arced (e.g., circular) path surrounding iris-shaped marking 80, and target-marker 98 may mark this path.

In response to viewing image 104, the user may ascertain whether the image processing of the controller requires correction, by comparing the positions of target-markers 96 to the expected positions of these target-markers. For example, if all the target points were supposed to be at a uniform distance from the edge of iris-shaped marking 80 but target-marker 98 is not at a uniform distance from the edge (i.e., the center of target-marker 98 does not coincide with the center of the iris-shaped marking), the user may ascertain that the image processing requires correction.

The user may also ascertain, in response to viewing image 104, whether calibration of the beam-directing elements is required. For example, if irradiated locations 76 are offset from target- marker 98 as shown in FIG. 5, the user may ascertain that such calibration is required.

To calibrate the beam-directing elements, the user may iteratively adjust one or more relevant parameters of the system and repeat the testing procedure (using any required number of cards 22) until the irradiated locations coincide with the target points to within a given level of tolerance.

For example, for embodiments in which the beam-directing elements comprise galvo mirrors 50 (FIG. 1), the controller may control the galvo mirrors by inputting a pair of voltages Vₓ and V_{y} to the mirrors, thereby causing the mirrors to direct the beam to the coordinates (bₓ+mₓ*Vₓ, b_{y}+m_{y}*V_{y}) in the FOV of the camera, where bₓ, b_{y}, mₓ, and my are adjustable parameters. During the calibration, the user may iteratively adjust any one or more of these adjustable parameters.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system, comprising:
a radiation source, configured to emit beams of radiation;
one or more beam-directing elements, configured to direct the beams;
a card configured to undergo a permanent change in appearance at sites on the card on which the beams impinge;
a camera, configured to acquire one or more images of the card; and
a controller, configured to:
process the images, and
in response to processing the images, control the beam-directing elements so as to direct the beams at one or more target points in a field of view (FOV) of the camera, thereby causing the appearance of the card to change at one or more irradiated locations on the card.

2. The system according to claim 1, wherein the card comprises a polymer.

3. The system according to claim 1, wherein the card comprises transparent glass.

4. The system according to claim 1, wherein the change in appearance includes a change in color.

5. The system according to claim 4, wherein the card comprises a photosensitive dye configured to undergo the change in color in response to the beams of radiation.

6. The system according to claim 4, wherein the card comprises a temperature-sensitive material configured to undergo the change in color in response to being heated by the beams of radiation.

7. The system according to claim 1, wherein the card is configured to undergo the change in appearance by virtue of the beams forming respective holes at the sites.

8. The system according to claim 1, wherein the controller is further configured to move the camera with respect to the card between acquisitions of the images.

9. The system according to claim 1, further comprising a jig configured to move the card with respect to the camera between acquisitions of the images.

10. The system according to claim 1, further comprising:
an optical unit; and
an XYZ stage unit comprising a control mechanism,
wherein the optical unit comprises the camera and is mounted onto the XYZ stage unit so as to be moveable by a user, using the control mechanism, between acquisitions of the images.

11. The system according to claim 1,
wherein the card comprises one or more markings, and
wherein, for each of the images, the controller is configured to:
identify at least one of the markings in the image, and
control the beam-directing elements in response to identifying the at least one of the markings.

12. The system according to claim 11, wherein, for each of the images, the controller is configured to control the beam-directing elements so as to direct a respective one of the beams at one of the identified markings.

13. The system according to claim 11,
wherein the markings include an iris-shaped marking that simulates a human iris with respect to shape, and
wherein, for each of the images, the controller is configured to:
identify the iris-shaped marking in the image,
compute a respective one of the target points with reference to the iris-shaped marking, and
control the beam-directing elements so as to direct a respective one of the beams at the computed one of the target points.

14. The system according to claim 13,
wherein a background of the card surrounding the iris-shaped marking has a background appearance, and
wherein, at at least one location along a perimeter of the iris-shaped marking, a transition between the background appearance and an appearance of the iris-shaped marking occurs over at least 0.1 mm.

15. The system according to claim 13,
wherein a background of the card surrounding the iris-shaped marking has a background appearance, and
wherein, at at least one location along a perimeter of the iris-shaped marking, a transition between the background appearance and an appearance of the iris-shaped marking occurs over less than 0.1 mm.

16. The system according to claim 1, wherein the controller is further configured to:
identify the irradiated locations in another image of the card,
in response to identifying the irradiated locations, compute a distance between one of the irradiated locations and the target point at which the beam that impinged on the irradiated location was directed, and
communicate an output in response to the distance.

17. The system according to claim 1, wherein the controller is further configured to:
prior to controlling the beam-directing elements, display another image of the card with one or more overlaid target-markers,
receive, from a user, an adjustment of respective positions of the overlaid target-markers, and
define the target points in response to the adjusted positions.

18. The system according to claim 1, wherein the controller is further configured to display another image of the card, which shows the irradiated locations, with one or more overlaid target-markers at the target points.

19. The system according to claim 18, wherein the card comprises an iris-shaped marking that simulates a human iris with respect to shape, and wherein the overlaid target-markers include an arced target-marker surrounding the iris-shaped marking and passing through the target points.

20. A method, comprising:
coupling a card, which is configured to undergo a permanent change in appearance at sites on the card on which beams of radiation impinge, to a jig; and
by inputting a command to a controller, initiating a testing procedure during which the controller:
processes one or more images of the card acquired by a camera while the card is coupled to the jig, and
in response to processing the images, controls one or more beam-directing elements so as to direct the beams at one or more target points in a field of view (FOV) of the camera, thereby causing the appearance of the card to change at one or more irradiated locations on the card.

21. The method according to claim 20, wherein the card includes a polymer.

22. The method according to claim 20, wherein the card includes transparent glass.

23. The method according to claim 20, wherein the change in appearance includes a change in color.

24. The method according to claim 23, wherein the card includes a photosensitive dye configured to undergo the change in color in response to the beams of radiation.

25. The method according to claim 23, wherein the card includes a temperature-sensitive material configured to undergo the change in color in response to being heated by the beams of radiation.

26. The method according to claim 20, wherein the card is configured to undergo the change in appearance by virtue of the beams forming respective holes at the sites.

27. The method according to claim 20, wherein, during the testing procedure, the controller moves the camera with respect to the card between acquisitions of the images.

28. The method according to claim 20, wherein, during the testing procedure, the jig moves the card with respect to the camera between acquisitions of the images.

29. The method according to claim 20,
wherein an optical unit includes the camera and is mounted onto an XYZ stage unit including a control mechanism, and
wherein the method further comprises, using the control mechanism, moving the optical unit between acquisitions of the images.

30. The method according to claim 20,
wherein the card include one or more markings, and wherein, for each of the images, the controller:
identifies at least one of the markings in the image, and
controls the beam-directing elements in response to identifying the at least one of the markings.

31. The method according to claim 30, wherein, for each of the images, the controller controls the beam-directing elements so as to direct a respective one of the beams at one of the identified markings.

32. The method according to claim 30,
wherein the markings include an iris-shaped marking that simulates a human iris with respect to shape, and
wherein, for each of the images, the controller:
identifies the iris-shaped marking in the image,
computes a respective one of the target points with reference to the iris-shaped marking, and
controls the beam-directing elements so as to direct a respective one of the beams at the computed one of the target points.

33. The method according to claim 32,
wherein a background of the card surrounding the iris-shaped marking has a background appearance, and
wherein, at at least one location along a perimeter of the iris-shaped marking, a transition between the background appearance and an appearance of the iris-shaped marking occurs over at least 0.1 mm.

34. The method according to claim 32,
wherein a background of the card surrounding the iris-shaped marking has a background appearance, and
wherein, at at least one location along a perimeter of the iris-shaped marking, a transition between the background appearance and an appearance of the iris-shaped marking occurs over less than 0.1 mm.

35. The method according to claim 20, wherein, during the testing procedure, the controller:
identifies the irradiated locations in another image of the card,
in response to identifying the irradiated locations, computes a distance between one of the irradiated locations and the target point at which the beam that impinged on the irradiated location was directed, and
communicates an output in response to the distance.

36. The method according to claim 20, further comprising, prior to initiating the testing procedure, adjusting respective positions of one or more target-markers overlaid on another image of the card,
wherein, during the testing procedure, the controller define the target points in response to the adjusted positions.

37. The method according to claim 20, wherein, during the testing procedure, the controller displays another image of the card, which shows the irradiated locations, with one or more overlaid target-markers at the target points.

38. The method according to claim 37, wherein the card includes an iris-shaped marking that simulates a human iris with respect to shape, and wherein the overlaid target-markers include an arced target-marker surrounding the iris-shaped marking and passing through the target points.
